(19) United States
(12) Patent Application Publication
(10) Pub. No.: US 2026/0209464 A1
(43) Pub. Date: Jul. 23, 2026

(54) ELECTRO-FENTON DEPOLYMERIZATION OF WASTE PLASTICS INTO VALUE-ADDED CHEMICALS

(71) Applicant: Battelle Memorial Institute, Richland, WA (US)

(72) Inventors: Juan A. Lopez-Ruiz, Richland, WA (US); Bhanupriya Boruah, Richland, WA (US); Oliver Y. Gutiérrez Tinoco, Richland, WA (US)

(73) Assignee: Battelle Memorial Institute, Richland, WA (US)

(21) Appl. No.: 19/033,013

(22) Filed: Jan. 21, 2025

(51) Int. Cl. C08J 11/16; C07C 51/16; C07C 51/47

(52) U.S. Cl. CPC C08J 11/16; C07C 51/16; C07C 51/47; C08J 2323/06

(57) ABSTRACT

A method that includes functionalizing a waste polymer with sulfonate groups and Fe³⁺, introducing the functionalized waste polymer into an electrochemical cell, generating H₂O₂ and hydroxyl radicals (OH) in the electrochemical cell, and reacting the functionalized waste polymer with the hydroxyl radicals (OH) thereby depolymerizing the functionalized waste polymer in the electrochemical cell.

### BACKGROUND

Up to 75% of all polymers produced in the United States each year are discarded to landfills, which represents an irremediable loss of energy and a severe threat to the environment. Recycled plastics are usually utilized as fuels or reprocessed to low-quality materials. The large amounts of plastics available for recycling could be a useful source of carbon for refinery feedstocks and to produce value-added chemicals. Unfortunately, current recycling has not be able to efficiently recover the inherent value in plastics, especially polyolefins and polystyrene, which are remarkably chemically stable.

Existing chemical routes to convert polymers into smaller molecules rely on elevated temperatures (above 400° C.), which is associated with a low degree of control on product distribution and low yields of useful molecules. Processes reported in open and patent literature describe thermal gasification and pyrolysis or catalytic hydrocracking using zeolites or combinations of zeolites with supported metal catalysts. A recent development includes the conversion of polyolefins at relatively low temperatures over noble metals supported on perovskites. This report, however, relies on the use of melts, which is unpractical for the development of continuous processes. Other inventions rely on the addition of external solvents, including cases that are not chemically compatible with polymers, e.g., water. These approaches make the process costly and could lead to low product yield. Therefore, a process with a high level of control and is still needed for converting plastics into value-added products or useful refinery feedstocks.

### SUMMARY

Disclosed herein is a method comprising:
functionalizing a waste polymer with sulfonate groups and a Fenton reaction reagent;
introducing the functionalized waste polymer into an electrochemical cell;
generating H₂O₂ and hydroxyl radicals (OH) in the electrochemical cell; and
reacting the functionalized waste polymer with the hydroxyl radicals (OH) thereby depolymerizing the functionalized waste polymer in the electrochemical cell.

Also disclosed herein is a method comprising:
introducing sulfonate- and Fe³⁺-functionalized waste polymer into an electrochemical reactor; and
performing a Fenton reaction in the electrochemical reactor thereby depolymerizing the functionalized waste polymer in the electrochemical reactor.

The foregoing will become more apparent from the following detailed description, which proceeds with reference to the accompanying figures.

### BRIEF DESCRIPTION OF THE DRAWINGS

FIG. 1. Carbon (C), hydrogen (H), sulfur(S) and iron (Fe) content in treated polymers namely polyethylene (PE), polypropylene (PP), polystyrene (PS) and polyvinylchloride (PVC) named as Polymer-SO₃—Fe obtained from CHS analyzer and ICP-MS. Polymer treatment procedure includes grafting sulfonate group and iron on polymer backbone.

FIG. 2. XPS narrow scan regions of the iron (Fe) 2p, carbon (C) 1s, oxygen (O) 1s, and sulfur(S) 2p regions for treated polyethylene (PE-SO₃—Fe).

FIG. 3. XPS narrow scan regions of the iron (Fe) 2p, carbon (C) 1s, oxygen (O) 1s, and sulfur(S) 2p regions for treated polypropylene (PP-SO₃—Fe).

FIG. 4. XPS narrow scan regions of the iron (Fe) 2p, carbon (C) 1s, oxygen (O) 1s, and sulfur(S) 2p regions for treated polystyrene (PS-SO₃—Fe).

FIG. 5. XPS narrow scan regions of the iron (Fe) 2p, carbon (C) 1s, oxygen (O) 1s, and sulfur(S) 2p regions for treated polyvinylchloride (PVC-SO₃—Fe).

FIG. 6. Schematic of a divided batch cell used for plastic upcycling via electro Fenton process. The reaction conditions were Pd on carbon felt as cathode, Pt wire as the counter electrode, Ag/AgCl as the reference electrode, 80 mL of electrolyte 0.1 M Na₂SO₄ in each anode and cathode compartment, 100 mg of substrate (treated polymers), air flow at 50 mL/min, room temperature and atmospheric pressure.

FIG. 7. Cyclic voltammetry for Pd 1 wt. % on Carbon felt showing applied potential regions for different reactions. H₂O₂ generation region is shown between 0.150 V to 0.670 V vs RHE. The reaction conditions were Pd on carbon felt as cathode, Pt wire as the counter electrode, Ag/AgCl as the reference electrode, 80 mL of electrolyte 0.1 M Na₂SO₄ in each anode and cathode compartment, air flow at 50 mL/min, room temperature and atmospheric pressure.

FIG. 8. Effect of cathode half-cell potential for H₂O₂ generation for 2 h. The reaction conditions were Pd on carbon felt as cathode, Pt wire as the counter electrode, Ag/AgCl as the reference electrode, 80 mL of electrolyte 0.1 M Na₂SO₄ in each anode and cathode compartment, air flow at 50 mL/min, room temperature and atmospheric pressure.

FIG. 9. Chronoamperometry plot for 20 hours at 0.45 V vs RHE without any polymer and with treated polymers polyethylene (PE), polypropylene (PP), polystyrene (PS), and polyvinylchloride (PVC). The reaction conditions were Pd on carbon felt as cathode, Pt wire as the counter electrode, Ag/AgCl as the reference electrode, 80 mL of electrolyte 0.1 M Na₂SO₄ in each anode and cathode compartment, 100 mg of substrate (treated polymers), air flow at 50 mL/min, room temperature and atmospheric pressure.

FIG. 10. Conversion of treated polyethylene (PE), polypropylene (PP), polystyrene (PS), and polyvinylchloride (PVC) after Fenton and Electro-Fenton process. Fenton process was performed with external addition of H₂O₂ 33,000 ppm. The reaction conditions for Electro Fenton were Pd on carbon felt as cathode at 0.45 V vs RHE that generated ≈10 ppm H₂O₂, Pt wire as the counter electrode, Ag/AgCl as the reference electrode, air flow at 50 mL/min. Both processes were carried out for 20 h, in 80 mL 0.1 M Na₂SO₄ solvent/electrolyte and 100 mg substrate (treated polymers), at room temperature and atmospheric pressure.

FIG. 11. Product distribution and selectivity after Fenton and e-Fenton processes for polymer conversion. Fenton process was performed with external addition of 33,000 ppm H₂O₂. The reaction conditions for Electro Fenton were Pd on carbon felt as cathode at 0.45 V vs RHE that generated 3 to 10 ppm H₂O₂, Pt wire as the counter electrode, Ag/AgCl as the reference electrode, air flow at 50 mL/min. Both processes were carried out for 20 h, in 80 mL 0.1 M Na₂SO₄ solvent/electrolyte and 100 mg substrate (treated polymers), at room temperature and atmospheric pressure.

FIG. 12. SEM images of polyethylene (PE) pristine, PE after treatment (PE-SO₃—Fe) and PE-SO₃—Fe after electro Fenton reaction. Cavities 2.59±1.97 μm observed after electro Fenton. SEM images taken at a magnification of 1000×, 2 kV accelerating voltage and 5 mm working distance.

FIG. 13. SEM images of polypropylene (PP) pristine, PP after treatment (PP-SO₃—Fe) and PP-SO₃—Fe after electro Fenton reaction. Cavities 1.57±1.87 μm observed after electro Fenton. SEM images taken at a magnification of 1000×, 2 kV accelerating voltage and 5 mm working distance.

FIG. 14. SEM images of polystyrene (PS) pristine, PS after treatment (PS-SO₃—Fe) and PS-SO₃—Fe after electro Fenton reaction. Cavities 4.20±1.94 μm observed after electro Fenton. SEM images taken at a magnification of 1000×, 2 kV accelerating voltage and 5 mm working distance.

FIG. 15. SEM images of polyvinylchloride (PVC) pristine, PVC after treatment (PVC-SO₃—Fe) and PVC-SO₃—Fe after electro Fenton reaction. Cavities 3.61±1.80 μm observed after electro Fenton. SEM images taken at a magnification of 1000×, 2 kV accelerating voltage and 5 mm working distance.

FIG. 16. Fourier transform infrared (FTIR) characterization of the polyethylene (PE), before and after treatment, indicating the introduction of sulfonate functional groups into the polymer material. Pretreatment procedure includes grafting sulfonate and iron groups on polymer backbone.

FIG. 17. Atomic % of carbon, sulfur, iron and oxygen content in treated polymers namely polyethylene (PE), polypropylene (PP), polystyrene (PS), and polyvinylchloride (PVC) named as Polymer-SO₃—Fe obtained from XPS. Polymer treatment procedure includes grafting sulfonate group and iron on polymer backbone.

FIG. 18. Cavity diameter observed from SEM images in polyethylene (PE), polypropylene (PP), polystyrene (PS), and polyvinylchloride (PVC) before treatment, after treatment and after electro Fenton reaction.

### DETAILED DESCRIPTION

### Overview

Disclosed herein is an electrochemical process to convert waste polymers, particularly polyolefins such as polyethylene (PE) and polypropylene (PP), into mono- and di-carboxylic acids via the electro-Fenton reaction at room temperature and atmospheric pressure in an electrochemical cell. The waste polymer (e.g., PE) is pretreated prior to undergoing electro-Fenton depolymerization. During the pretreatment, the waste polymers are functionalized with sulfonate groups followed by functionalization with Fe³⁺ coordinated with the sulfonate group. The electrochemical cell generates H₂O₂ for the electro-Fenton reaction via oxygen (O₂) reduction at the cathode (O₂+2H⁺+2e⁻→H₂O₂) and followed by the H₂O oxidation at the anode (2H₂O→O₂+4H⁺+4e⁻). The in situ generated H₂O₂ reacts with the Fe³⁺ grafted onto the polymer and in situ produces hydroxyl radicals (OH) via the Fenton reaction (H₂O₂+Fe²⁺→OH⁻+·OH+Fe³⁺) that depolymerize the treated waste polymers. The Fe³⁺ is then reduced into Fe²⁺ at the cathode (Fe³⁺+2e⁻→Fe²⁺).

The electrochemical depolymerization process allows for a continuous conversion of polyolefins into smaller chain mono- and di-carboxylic acids that can be separated following standard procedures. This enables the use of discarded polymers that would be otherwise be burned or lost in landfills, to generate value as chemicals. The process can be used in diluted (waste) water streams and can be applied to remove microplastics. The process does not require the purchase and storage of external agents (e.g., H₂, H₂O₂) to depolymerize the polyolefins as they are generated in situ; hence, it is inherently a safer operation as well as more amenable to distributed processing and electrification with renewable energy. In addition, the mixture adds one more element of control (i.e., concentration of H₂O₂) to the conversion of polymers, which can be used to tune the rates of the conversion and the selectivity. Furthermore, H₂ is generated as a byproduct with the excess electrons and protons at the cathode via the hydrogen evolution reaction (HER: 2H⁺+2e⁻→H₂), which can be sold or used on site for heat and electrical power generation.

The process can be utilized for converting waste plastic from polymer manufacturing plants as well as wastewater management companies and landfills. Incineration and pyrolysis are the most common methods to process waste plastics; otherwise, they are disposed of and accumulated in landfills. Because the process disclosed herein can be performed at near room temperature and atmospheric pressure, it is amenable for distributed processing at the generation site as opposed to transporting to a centralized location. Additionally, unlike thermal plastics, the process disclosed herein can be used to treat microplastics in water and can be potentially incorporated at the generation points (e.g., households, plastic processing plants, textiles) to treat them before they are released and diluted in the environment.

The waste plastics can be any solid polymeric material that has been discarded after use or after the intended use is over. The waste plastics can also be undesired excess byproduct of a manufacturing or packaging facility. In certain examples, the waste polymer is a polyolefin, particularly polyethylene (PE) or polypropylene (PP). PE and PP represent the majority of post-consumer plastic waste, especially in packaging. Additional polymers such as polystyrene (PS) and polyvinylchloride (PVC) can also be decomposed using this process.

### Pretreatment

The waste polymer (e.g., PE) is pretreated prior to undergoing electro-Fenton depolymerization. The polymers are first functionalized with sulfonate groups followed by grafting with co-catalyst(s) (e.g., FeCl₃).

Illustrative repeating unit structures for PE and PP showing the grafted sulfonate and iron are shown below.

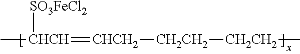

### Polyethylene-SO₃—Fe

wherein x is 100 to 100,000, more particularly 100 to 10,000

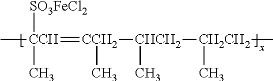

### Polypropylene-SO₃—Fe

x is 250 to 7,000, more particularly 250 to 5,000.

The solid waste polymer is initially dissolved in a solvent(s). For example, polymer PE or PP about 1 g is dissolved in 25 mL of solvent (possible solvents: 1,2 dichloroethane, 1,1,2,2-tetrachloroethane or chloroform) with stirring (200 rpm) under reflux at 65° C.

Chemical introduction of hydrophilic groups (such as sulfonate group) to the polymer backbone helps in activating the polymer. Thus, sulfonate grafting helps in activating the inert polymer. The sulfonate content can range from 5 to 15 wt %.

For sulfonation, 1 g of polymer is dissolved in 25 mL of chloroform with stirring (200 rpm) under reflux at 65° C. Then, a 15 mL mixture of chlorosulfuric acid (5 mL) and dichloromethane (10 mL) is added dropwise over 30 min and then stirred for additional 2 h. The solvent is removed under reduced pressure. Other sulfonating agents can be used such as sulfuric acid, chlorosulfuric acid, and trimethylsilyl chlorosulfonate.

FeCl₃ is also grafted onto the polymer backbone. Only Fe³⁺ and Fe²⁺ is used in the e-Fenton reaction. Fe³⁺ which serves as a catalyst reacts with H₂O₂ to generate Fe²⁺ which then reacts with the generated H₂O₂ to produce ·OH radicals and regenerate Fe³⁺. The generated ·OH radicals attack the sulfonated polymers to partially oxidize them into low molecular weight organic acids (e.g., oxalic acid, formic acid, acetic acid) or fully oxidize them into carbon dioxide. The iron content on the polymer after grafting ranges from 1 to 7 wt. %.

After the sulfonation step, 100 mL of 0.75 M FeCl₃ water solution is added to the mixture and stirred overnight. The black precipitate obtained by filtration is washed with DI water until the filtrate had a neutral pH. The solids are then oven-dried at 80° C. overnight and then utilized for the Fenton and Electro-Fenton experiments. These solids are now polymers with Fe³⁺ grafted through coordination with sulfonate groups. List of possible iron addition agents: ferric chloride, ferrous sulfate, ferrous sulfate heptahydrate, pentahydrated ferric sulfate. Other Fenton reagents such as Ag, Ce, Cu, or Mn can be used instead of Fe.

### Electro-Fenton Reaction in an Electrochemical Cell

The treated polymer is then introduced into an electrochemical cell that can generate H₂O₂. In certain examples, the treated polymer can be introduced into the cell in a semi-batch or continuous process.

In the semi-batch example, the treated polymer is loaded into an electrochemical reactor containing the electrolyte(s) and the electrodes and the reactor is run until the polymer is fully decomposed, and then more polymer is loaded into the reaction again.

Because the electrochemical cell current is directly proportional to the polymer decomposition rate, the process can be automated to add more or less polymer as a function of the current changes. The polymer may be continuously added at the same speed as the polymer decomposition rate.

In the continuous example, the treated polymer and the electrolyte are premixed and the introduced into a continuous flow electrolyzer. The ratio of polymer to electrolyte can be adjusted to enable the electrocatalysis and avoid reactor plugging.

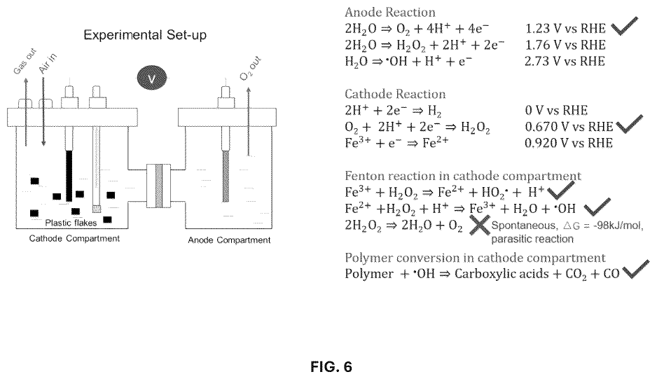

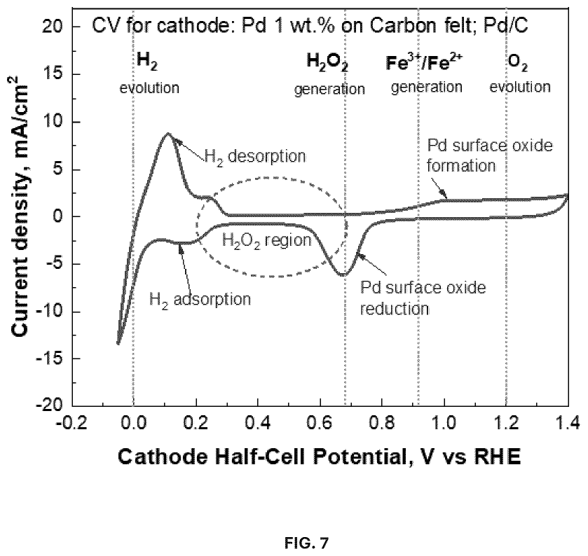

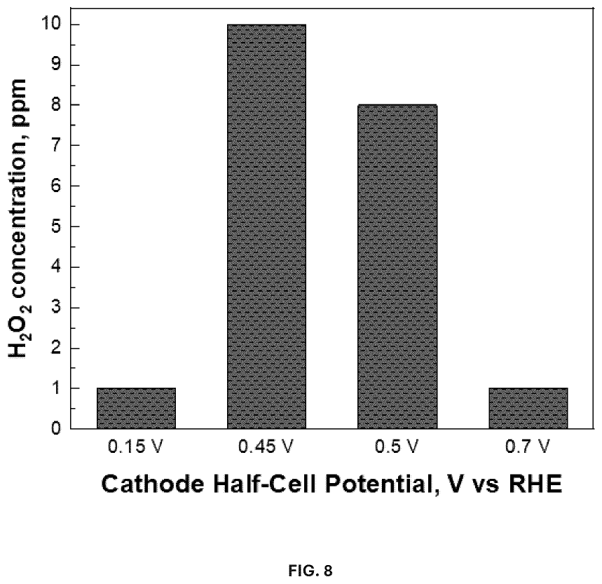

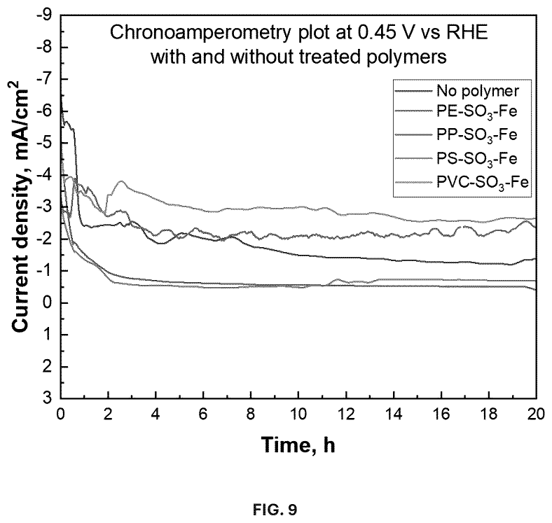

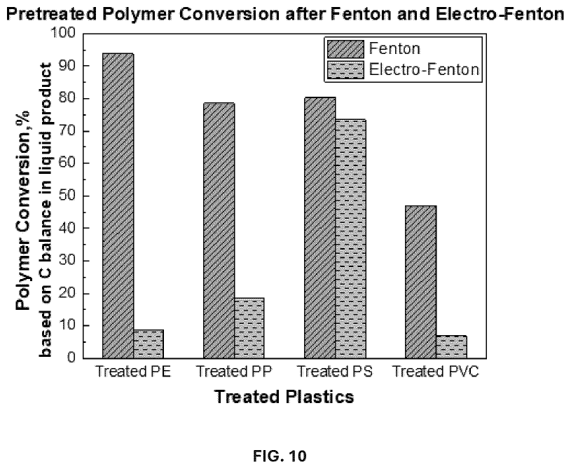

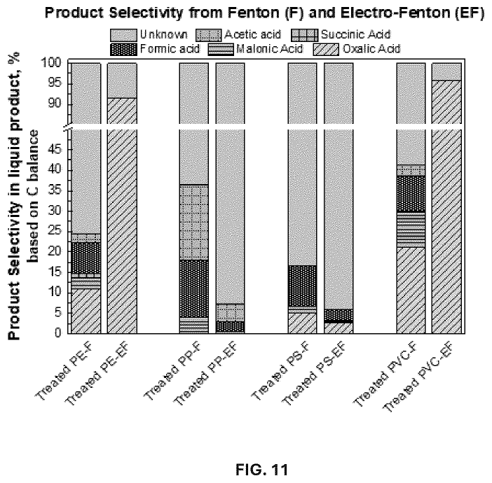

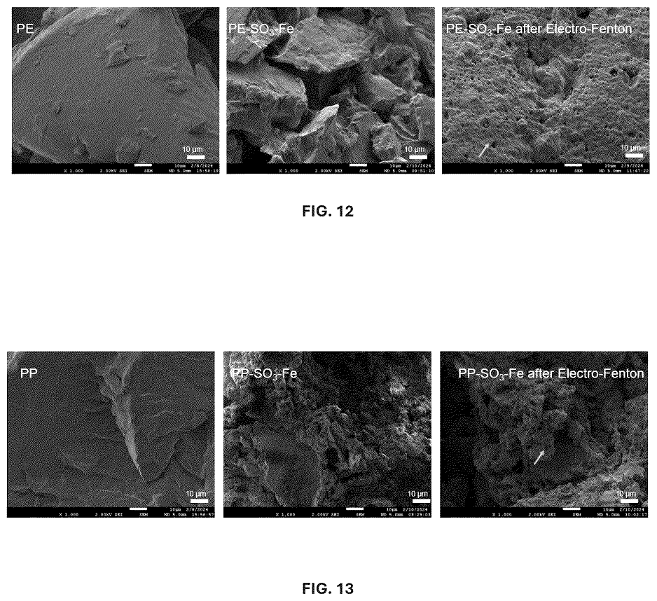

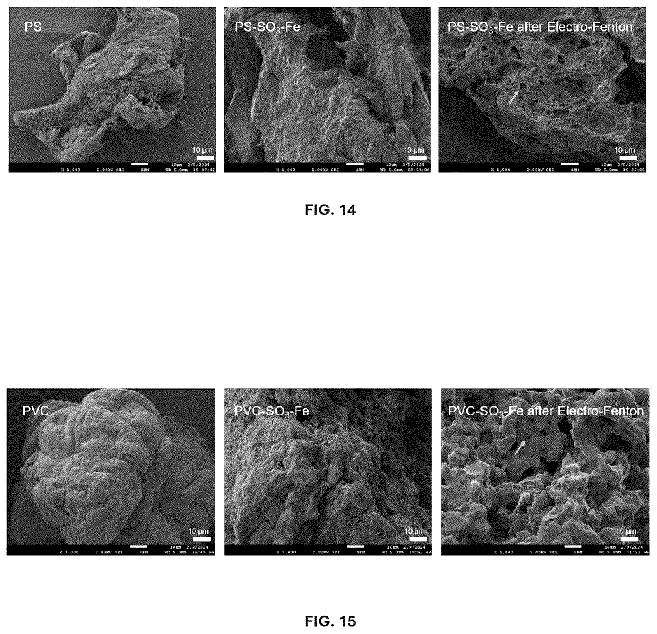

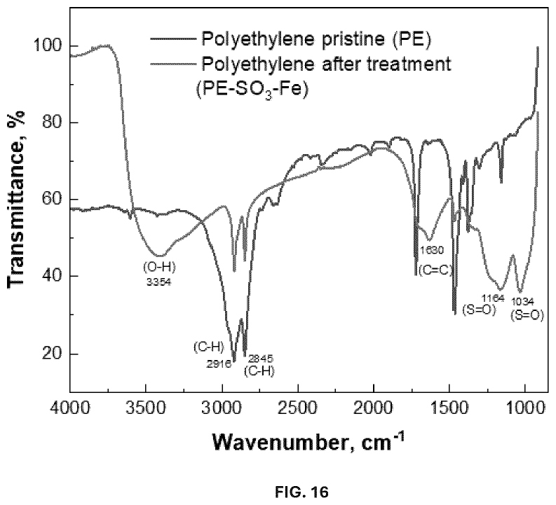

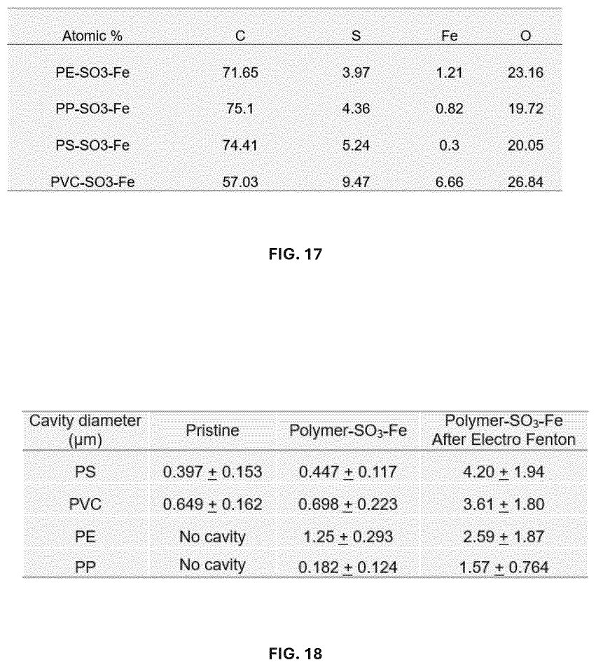

The electrochemical cell includes a cathode compartment that includes a cathode that in-situ generates H₂O₂ via the oxygen reduction reaction (ORR: O₂+2e⁻+2H⁺→H₂O₂) and H₂ via the hydrogen evolution reaction (HER: 2e⁻+2H⁺→H₂). The Fe³⁺ co-catalyst can also be regenerated in the cathode compartment (Fe³⁺+e⁻→Fe²⁺). The electrochemical cell also includes an anode that may be used for performing the oxygen evolution reaction (OER: 2H₂O→O₂+4e⁻+4H). Hydroxyl radicals (·OH) generated in situ via the Fenton reaction depolymerize the polymer. The excess H⁺ and e⁻ are recombined into H₂. An example of an electrochemical cell and the accompanying reactions is shown in FIG. 6.

Applying an external half-cell potential in the range of 0.15 V to 0.70 V vs RHE generates −10 to −0.10 mA/cm² which generates H₂O₂ in the range of 0.5 to 10 ppm. The generated H₂O₂ reacts with Fe³⁺ to generate ·OH, which are powerful oxidizing agents which attacks the polymer that led to polymer decomposition

The cathode can include a graphite rod, carbon felt or carbon paper. The cathode also can include a catalyst. Illustrative catalysts include Pd nanoparticles, carbon nanotubes, Co nanoparticles, CoSe₂, NiSe₂ deposited on a conductive support such as carbon felt or carbon paper.

The anode can include graphite or a metal (e.g., titanium, tantalum, nickel) film or foam support coated with metal oxides such as RuO₂, IrO₂, NiO, MnO, PbO₂, Co₃O₄ on conductive support.

The anode and cathode can be regenerated on site (if needed) by switching the potentials from negative to positive and vice versa.

The electrochemical cell also includes an aqueous electrolyte. Illustrative electrolytes include 0.1 to 3.0 M Na₂SO₄, H₂SO₄, NaHSO₄/Na₂SO₄ buffer, HNO₃, NaCl, or HClO₄ solutions.

The electrochemical cell also separates the anode and the cathode compartment using a physical barrier such as membranes (e.g., Nafion, Sustainion, glass fibers) or frits (e.g., glass). The role of the physical barriers is to inhibit crossover of reagents between the anode and cathode and mitigate undesirable reactions.

Oxygen (O₂) is introduced into the cathode compartment. In certain examples, air is introduced into the cathode compartment as the oxygen source. An aqueous electrolyte introduced into the anode compartment. Oxygen (produced by the OER reaction) exits the anode compartment. The main gas compounds at the cathode are CO and CO₂, consistent with the decomposition of the polymers.

Illustrative products produced by the process disclosed herein include oxalic acid, malonic acid, acetic acid, formic acid, succinic acid, CO, and CO₂. In certain examples, the process is selective for producing oxalic acid. In certain examples, the process is 100% selective for producing oxalic acid.

The products (e.g., carboxylic acids) are soluble in the electrolyte and may be removed from the electrochemical cell. For example, the carboxylic acids(s) products can be removed from the cathode compartment by separating the products from the aqueous electrolyte. The separation could be accomplished via a membrane separation or electrodialysis. If there is no need for the carboxylic acids, a second electrochemical oxidation can be performed to make H₂ or other products from the acids that can be easily separated from the aqueous electrolyte.

### EXAMPLES

Polymer Pretreatment—Preparation of PE Grafted with SO₃ and Iron

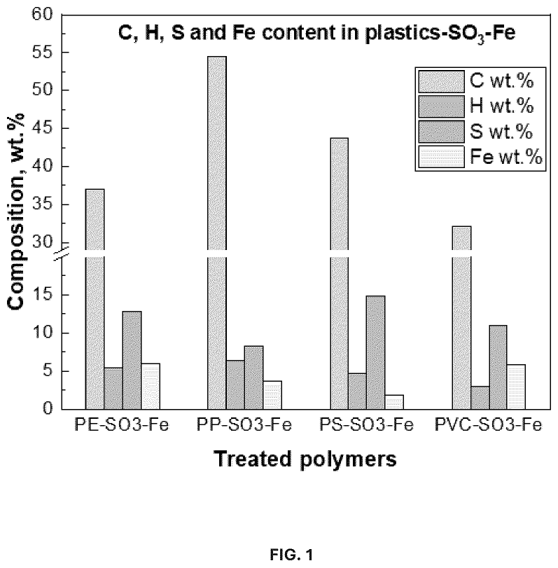

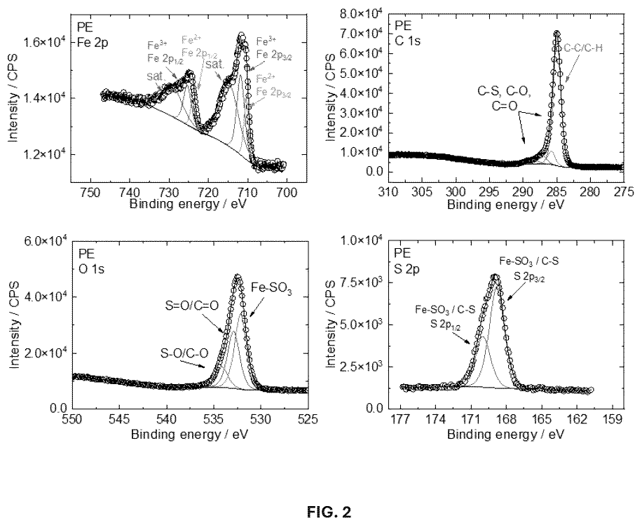

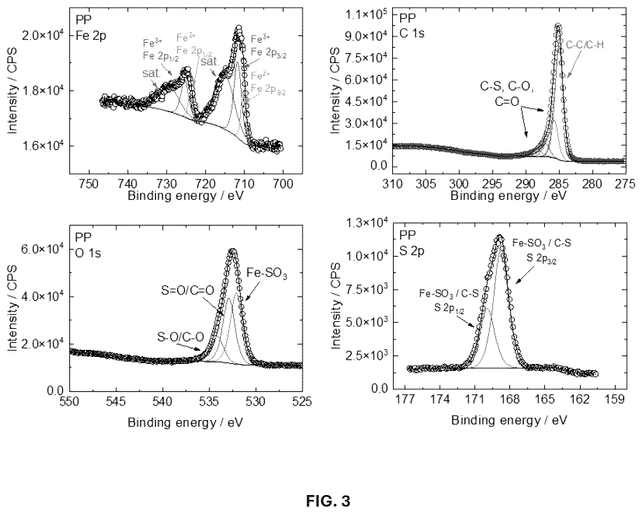

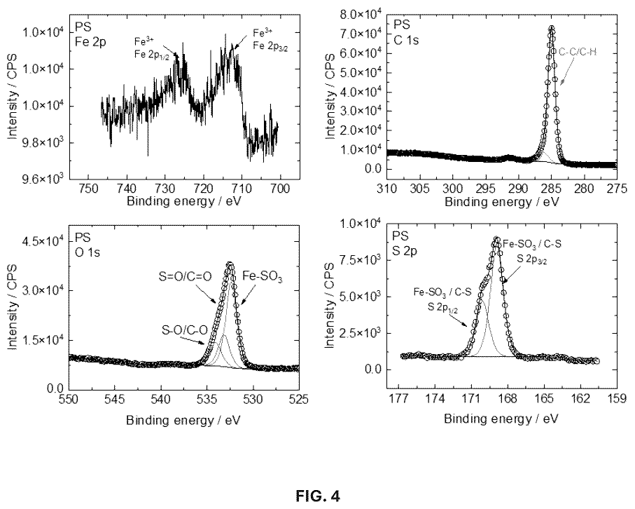

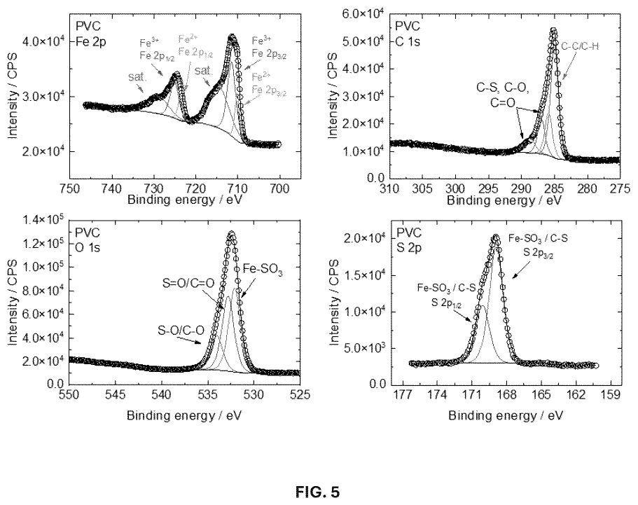

Low density polyethylene (LDPE) was ground into powder with particle sizes 125-250 μm. The PE (~1 g) as then dissolved in 25 mL of chloroform with stirring (200 rpm) under reflux at 65° C. A mixture of chlorosulfuric acid (12.5 vol %) and dichloromethane (15 mL, 1:2 v/v) was added drop-wise over 30 min and then stirred for additional 2 h. The solvent was removed under reduced pressure. FeCl₃ solution (0.75 M, 100 mL) was added to the mixture, and stirred overnight. The black precipitate obtained by filtration was washed with DI water until the filtrate had a neutral pH. The solids were then oven-dried at 80° C. overnight and then utilized for electro-Fenton experiments. The resulting treated polymer is referred to as “PE-SO₃—Fe”. The FTIR analysis of the treated polymer is shown in FIG. 1. Carbon and sulfur analysis indicated the PE-SO₃—Fe contained approximately 36% C and approximately 12% sulfur.

### Electro-Fenton Treatment

The electro-Fenton (EF) process was performed in a two-compartment electrochemical cell. The anode compartment consisted of a working electrode (Pd catalyst 1 wt. % on Carbon felt); Ag/AgCl as the reference electrode; and provision for air bubbling at 500 mL/min. The cathode compartment consisted of a counter electrode (Pt mesh). Both the compartments were filled with 70 mL of 0.1M Na₂SO₄ pH 2.6. Both the compartments were filled with 70 mL of 0.1 M Na₂SO₄ in water (i.e., the electrolyte) with a resulting pH of 2.6. Prior to conducting the EF process, the anode compartment was purged with Ar for 20 mins and then with air for 1 h. PE-SO₃—Fe was then introduced to the anode compartment. The EF process was then carried out at different applied potentials to generate H₂O₂ and the degrade PE under constant stirring at 500 rpm and air bubbling.

The working electrode was prepared by cleaning carbon felt size (1×3 cm²) with acetone and DI water. The clean C felt was then plasma treated under O₂ at 1 torr for 15 mins. The weight and the pore volume of the treated C felt was measured (~11.7 mL/g). The desired amount of Pd nitrate (4 mg per 0.19 g of C felt) was dissolved in water and drop cast on the C felt. The C felt was loaded with metal precursor is then dried overnight at 80° C. The electrode was thermally treated under N₂ at 180° C., 100 mL/min for 3 h to decompose the precursor followed by metal reduction under 5% H₂/N₂ at 100 mL/min for 3 h, 250° C.

In view of the many possible embodiments to which the principles of the disclosed invention may be applied, it should be recognized that the illustrated embodiments are only preferred examples of the invention and should not be taken as limiting the scope of the invention.

What is claimed is:

1. A method comprising:
functionalizing a waste polymer with sulfonate groups and a Fenton reaction reagent;
introducing the functionalized waste polymer into an electrochemical cell;
generating H₂O₂ and hydroxyl radicals (·OH) in the electrochemical cell; and
reacting the functionalized waste polymer with the hydroxyl radicals (·OH) thereby depolymerizing the functionalized waste polymer in the electrochemical cell.

2. The method of claim 1, wherein the waste polymer comprises waste polyolefin.

3. The method of claim 1, wherein the waste polymer is waste polyethylene or waste polypropylene, or a mixture thereof.

4. The method of claim 1, comprising functionalizing the waste polymer with sulfonate groups and then functionalizing the sulfonated waste polymer with the Fenton reaction reagent.

5. The method of claim 1, wherein functionalizing the waste polymer with sulfonate groups comprises reacting the waste polymer with sulfuric acid, chlorosulfuric acid, or trimethylsilyl chlorosulfonate.

6. The method of claim 4, wherein the sulfonated waste polymer has a sulfonate content of 5 wt % to 15 wt %.

7. The method of claim 1, wherein the Fenton reaction reagent is Fe³⁺.

8. The method of claim 4, wherein the Fenton reaction reagent is Fe³⁺.

9. The method of claim 8, wherein functionalizing the waste polymer with Fe³⁺ comprises reacting the sulfonated waste polymer with ferric chloride, ferrous sulfate, ferrous sulfate heptahydrate or pentahydrated ferric sulfate.

10. The method of claim 7, wherein the functionalized waste polymer has 1 wt % to 7 wt % Fe.

11. The method of claim 1, wherein the electrochemical cell includes a cathode comprising carbon felt or carbon paper.

12. The method of claim 11, wherein the cathode further comprises a catalyst comprising Pd nanoparticles, carbon nanotubes, Co nanoparticles, CoSe₂, or NiSe₂ disposed on the carbon felt or carbon paper.

13. The method of claim 1, wherein the electrochemical cell includes an anode comprising Ti foil or Ni foam.

14. The method of claim 13, wherein the electrochemical cell includes an anode comprising graphite rods RuO₂, IrO₂, NiO, MnO, PbO₂, or Co₃O₄ disposed on the Ti foil or Ni foam.

15. The method of claim 1, wherein the electrochemical cell includes an aqueous electrolyte comprising Na₂SO₄, H₂SO₄, NaHSO₄/Na₂SO₄ buffer, or HClO₄.

16. The method of claim 1, wherein the H₂O₂ is generated in a cathode compartment of the electrochemical cell.

17. The method of claim 16, wherein the H₂O₂ is generated via O₂+2e⁻+2H⁺→H₂O₂.

18. The method of claim 1, wherein the hydroxyl radicals (·OH) are generated via a Fenton reaction.

19. The method of claim 1, wherein depolymerizing the functionalized waste polymer produces at least one dicarboxylic acid.

20. The method of claim 1, wherein depolymerizing the functionalized waste polymer produces oxalic acid.

21. The method of claim 1, wherein depolymerizing the functionalized waste polymer selectively produces oxalic acid.

22. The method of claim 1, wherein air is introduced into a cathode compartment of the electrochemical cell, and an aqueous electrolyte is introduced into an anode compartment of the electrochemical cell.

23. The method of claim 1, further comprising dissolving solid waste polymer is in at least one solvent prior to the functionalizing.

24. The method of claim 19, there the at least one carboxylic acid product is removed from the electrochemical call by separating the least one carboxylic acid product from an aqueous electrolyte present in the electrochemical cell.

25. The method of claim 1, wherein the method produces H₂ as a by-product.

26. A method comprising:
introducing sulfonate- and Fe³⁺-functionalized waste polymer into an electrochemical reactor; and
performing a Fenton reaction in the electrochemical reactor thereby depolymerizing the functionalized waste polymer in the electrochemical reactor.

27. The method of claim 26, wherein the waste polymer is waste polyethylene or waste polypropylene; and depolymerizing the functionalized waste polymer produces at least one dicarboxylic acid.
